(19) United States
(12) Patent Application Publication
(10) Pub. No.: US 2022/0183587 A1
(43) Pub. Date: Jun. 16, 2022

(54) BREATH SAMPLING SYSTEM

(71) Applicant: Avisa Diagnostics, Inc., Santa Fe, NM (US)

(72) Inventors: David L. KARSHMER, Santa Fe, NM (US); Ramar Jette, Santa Fe, NM (US); John Maynard, Brookhaven, GA (US); Robert Alei, Santa Fe, NM (US)

(73) Assignee: Avisa Diagnostics, Inc., Santa Fe, NM (US)

(21) Appl. No.: 17/644,150

(22) Filed: Dec. 14, 2021

Related U.S. Application Data
(60) Provisional application No. 63/125,065, filed on Dec. 14, 2020.

(51) Int. Cl. A61B 5/083; A61B 5/097; A61B 10/00

(52) U.S. Cl. CPC A61B 5/0836; A61B 2010/0087; A61B 10/00; A61B 5/097

(57) ABSTRACT

A breath collection system, includes a vacuum reservoir; a vacuum connected to the vacuum reservoir; a breath collection reservoir configured to be located inside the vacuum reservoir during a breath collection and removed after a breath is collected from a patient; and a patient interface device connected to the breath collection reservoir to collect the breath from the patient.

### CROSS REFERENCE TO RELATED APPLICATIONS

This application claims the benefit of U.S. Provisional Patent Application No. 63/125,065, filed Dec. 14, 2020, which is hereby incorporated by reference for all purposes as if fully set forth herein.

### BACKGROUND

The invention is directed to a system and method of sampling a person's breath for detecting lung, gastrointestinal tract, and systemic infections. This system can be used with ambulatory or mechanically ventilated patients.

Early detection of whether a patient on a mechanical ventilator has a bacterial infection is important in providing suitable medical treatment for the patient and producing acceptable health outcomes. Methods of detecting the presence of bacteria in the body by measuring isotopically-labeled ratios of volatile gases have been reported (see, e.g., U.S. Pat. No. 7,717,857). In patients on ventilation, respiratory infections can cause significant increases in length and cost of care as well as increased morbidity. Ventilator Associated Pneumonia (VAP) is the most significant form of infection in ventilated patients who are at increased risk of infection due to compromised immune response. The pathogens at play in the VAP are often urease metabolizing bacteria which are detectable at an early point of infection using the inventions described in the following sections of this document.

Gastrointestinal and systemic infections can be detected using a urea breath test. What varies for these infections is that a drug tracer is given to the patient by different means (e.g., ingestion, IV, other) rather than by inhalation. Other than this the same breath sampling sequence occurs i.e., baseline breath sampling, delivery of drug tracer, and collection of second breath sample at a specified time post drug delivery.

### SUMMARY

To overcome the problems described above, preferred embodiments of the present disclosure provide a system and method of detecting respiratory and systemic infections in ventilated patients by delivering a drug i.e., ‘drug tracer’ to the ventilated patient that is metabolized by putative urease pathogens colonizing and infecting the mechanically ventilated patient. The metabolism of the drug tracer produces elevations in the abundance of ¹³CO₂ in specially collected breath samples.

The described process involves collection of a “baseline” breath sample(s) before introduction of the drug into the ventilated patient's respiratory airway, and single or multiple breath samples collected from the patient's respiratory tract at a selected period or periods after the completion of the drug delivery. Each breath sample can consist of one or more sequentially collected volumes that add up to a sample volume adequate for analysis, e.g., >50 ml. Comparing the abundance of ¹³CO₂ in collected sample(s) to an abundance of ¹³CO₂ in the baseline sample allows for the detection of urea metabolizing infections of interest. Also, described is an empty-able, multi-sample capable collection reservoir and a programmable collection system that allows for sequenced collection of multiple breath samples from the patient at appropriate times in the ventilator cycle. The collection of breath samples from ventilated patients for analysis of breath gases and micro particulates that are naturally occurring in the breath in association with infection and other health conditions. A method to determine the presence or absence of a bacterial infection in a patient on mechanical ventilation is provided.

In ventilated patients, the detection of infections can be difficult before the patient becomes symptomatic. A system for early detection of respiratory and other urease pathogen infection in patients allows for pre-symptomatic assessment of the presence and level of putative urease pathogens associated with Ventilator Associated Pneumonia in ventilated patients. In addition, collection of single or multiple breath samples with adequate levels of ¹³CO₂ for analysis is complicated by the operation of the ventilator and its delivery of refreshed air into the airway through which breath samples can be accessed.

The present disclosure describes delivery of a non-radioactive isotopically labeled drug as a tracer into the ventilated patient's airway or, otherwise systemically introduced, that is to be metabolized by putative urease pathogens to produce changes in the levels of ¹³CO₂ abundance and a measure called the Delta over Baseline which is commonly used to understand the change in ¹³CO₂ abundance in ratio to ¹²CO₂.

The delivery of the drug tracer to the patient is through (i) the ventilation airway by nebulization either at the connection of the ventilator circuit to the endotracheal tube through the use of a nebulizer adapter, or (ii) to specific locations in the respiratory anatomy by use of a drug delivery catheter or lumen in a specially purposed multi-lumen drug delivery and sample collection catheter, or (iii) or systemically (e.g., intravenously, via a nasal feeding tube, etc.).

The present disclosure describes the collection of a breath sample that provides samples that can successfully analyzed and provide information regarding the infection detection. The collection of breath samples according to disclosed embodiments prevents the significant dilution of the patient breath sample(s) and makes the assessment of changes in ¹³CO₂ abundance ratio changes possible. This mitigates circuit dilution of the exhaled breath and will also increase the measurable concentrations of other breath volatiles, and/or to localize the collection of breath gases to particular aspects or extents of the patient anatomy. This allows the use of multiple, time spaced samples in the detection of infections and their changes over time in response to medical therapy. Electronic sequencing of the delivery of the drug tracer and collection of the breath samples is performed so that the detection system and single use disposable (SUD) collection devices can be used to observe the change in ¹³CO₂ abundance at multiple periods over time.

The ventilator breath dilution effect has been demonstrated with ¹²CO₂ samples introduced into a ventilator circuit using a lung simulator with a CO₂ feed, a Siemens Maquet Servo-i ventilator/circuit, and a real time side stream ¹²CO₂ measurement instrument attached to the expiratory side of the ventilator circuit. CO₂ in the ventilator circuit was unmeasurable at a variety of flow rates and ventilator settings. As indicated by these findings, approaches are needed to optimize breath sample collection to minimize sample dilution during the ventilator cycle.

In an embodiment, a breath collection system, includes a vacuum reservoir; a vacuum connected to the vacuum reservoir; a breath collection reservoir configured to be located inside the vacuum reservoir during a breath collection and removed after a breath is collected from a patient; and a patient interface device connected to the breath collection reservoir to collect the breath from the patient.

The breath collection system can further include a housing that houses the vacuum reservoir and the breath collection reservoir. The vacuum can be a vacuum pump in the housing.

The breath collection system can further a user interface.

The breath collection system can further a biologic filter between the patient interface and the breath collection reservoir.

In an embodiment, the patient interface includes a nebulizer. In an embodiment, the patient interface includes a catheter.

The breath collection system can further include a regulator valve between the vacuum and the vacuum reservoir to regulate pressure in the vacuum reservoir. In an embodiment, the breath collection reservoir is a flexible bag. In an embodiment, the breath collection reservoir is a plurality of breath collection reservoirs. In an embodiment, the plurality of breath collection reservoirs includes a baseline breath collection reservoir and a sample breath collection reservoir.

The breath collection system can further include a pneumatic manifold between the vacuum and the vacuum reservoir and between the patient interface device and the breath collection reservoir.

The breath collection system can further include a pressure sensor in the vacuum reservoir.

In another embodiment, a breath collection device includes a vacuum reservoir; a baseline breath collection reservoir and a sample breath collection reservoir both configured to be located inside the vacuum reservoir and removed after breath samples are collected; a pneumatic manifold including a plurality of valves; a patient interface device to collect the breath from a patient connected to the baseline breath collection reservoir and the sample breath collection reservoir via a corresponding sample collection valve in the pneumatic manifold; and a vacuum connected to the vacuum reservoir via a vacuum valve in the pneumatic manifold.

In an embodiment, the plurality of valves includes 1 to n number of sample collection valves, and the 1 to n sample collection valves are connected to a corresponding baseline breath collection reservoir or a sample breath collection reservoir.

The breath collection device can further include a spectrometer valve in the pneumatic manifold and configured to direct the collected breath to a spectrometer. In an embodiment, the spectrometer valve is configured to connect the baseline breath collection reservoir and the sample breath collection reservoir to the spectrometer.

The above and other features, elements, characteristics, steps, and advantages of the present invention will become more apparent from the following detailed description of preferred embodiments of the present invention with reference to the attached drawings.

### BRIEF DESCRIPTION OF THE DRAWINGS

FIG. 1 is a block diagram of a breath sampling device according to an embodiment.

FIG. 2 is a block diagram of a breath sampling device according to another embodiment.

FIG. 3 is a flowchart showing exemplary steps in a breath test and the use of the breath sampling system according to another embodiment.

### DETAILED DESCRIPTION

A breath collection device and disposable bags are included in the breath collection components of the disclosed breath sampling system. The breath sampling system is used to detect urease pathogens in patients suspected of having respiratory tract infections. The breath sampling system can include an IR spectrometer that measures changes in the abundance ratio of ¹³CO₂ to ¹²CO₂ in breath samples.

The breath sampling device is capable of collecting breath samples from ambulatory, free breathing, patients as well as patients who are being mechanically ventilated. In both cases, breath samples are collected in valved, multilayer bags. The bags can be transported to the IR spectrometer for analysis or an integrated spectrometer can analyze breath sample without removing the bags from the breath sampling device.

In operation, a baseline breath is captured from the patient. This is followed by delivery of a drug tracer to the patient. A second breath is then collected sometime after the drug delivery and the breath samples are analyzed in the IR spectrometer.

Drug Delivery:

Drugs delivered to the patient using the respiratory infection detection system can be by nebulizer or catheter. Nebulized delivery can be accomplished using a mesh nebulizer adapted to the ventilator circuit proximal to the end of the ET tube closest to ventilator circuit. A mesh nebulizer delivery is an electronically motivated vibrating mesh nebulizer whose small volume drug delivery start and stop is electronically activated/sensed and can be controlled in direct relation to the ventilator cycle, the breath testing sequence, and the end of delivery of the drug dose. Jet nebulizer delivery is an air powered nebulizer that uses an electronically controlled compressor to provide a stream of air that entrains a drug in a small volume reservoir into the air stream and against a baffle generating a nebulized drug mist that is inhaled by the patient. The drug delivery start and stop can be electronically activated and can be controlled in direct relation to the breath testing sequence.

Drip or spray catheterization can deliver the drug to the location of a suspected infection using a catheter with a delivery lumen and an exit port or spray inducing feature at its tip. This catheter can also be a Multiport multi-lumen, multiport catheter including (i) a lumen to deliver a drug, (ii) a lumen to collect a breath sample(s), and (iii) a pressure measurement lumen/port.

Breath Sample Collection:

Location of Collection: Collection of breath gases at or slightly below the distal end of the EndoTracheal (ET) or Tracheotomy Tube is advantageous in ventilated patients because it mitigates the volume dilution of collected breath samples by the high bias flows of ambient air through the external ventilator circuit. Collection of breath gases from the respiratory anatomy below the endotracheal or tracheotomy tube can be accomplished by use of an indwelling sampling or suction catheter or other dedicated catheter where the catheter can include multiple sample collection ports or bores in the catheter tip to prevent occlusion of the catheter by placement of the tip against a tracheal tube or anatomical surface.

The catheter can also have a lumen that is attached to an external, reusable pressure transducer to measure patient airway pressures and detect the transition to the expiratory phase of ventilation and providing input to breath collection triggering. The end of the pressure sensing lumen should be at least 5 cm from the breath collection lumen bore at the catheter tip to prevent breath collection by the system, which can cause local pressure drops around the collection bore, from interrupting the measurement of general respiratory airway pressures.

Use of a multi-lumen catheter that has lumens for the delivery of a drug, a unique lumen(s) for the collection of respiratory gases, and a lumen for the measurement of airway pressure changes during the ventilator cycle. A steerable multi-lumen catheter can also be used for drug delivery to and sampling from the right or left lobes of the lung allowing more specific location of the urease pathogen colonization or infection in cases where a respiratory infection is suspected.

Collection of breath gases from the respiratory anatomy below the endotracheal or tracheotomy tube can be accomplished by collection using a specialized ET tube with sample collection port(s). This specially designed ET tube can have a bore occluding mechanism to temporarily occlude the air path between the ventilator circuit and the sampling space during breath sample collection.

A sequential collection of multiple small breath samples can be accumulated into a single sample for analysis. Breath is to be collected from the expiratory phase of the ventilator cycle to prevent volume dilution by incoming, ambient inspiratory gases. Due to the short duration of the expiratory phase, it can be useful to collect and accumulate multiple small samples for analysis. These smaller samples can be triggered using the airway pressures to be collected and transferred into the sample collection bag sequentially during the expiratory phases of ventilated breath.

Breath collections can be triggered using a breath sampling algorithm that determines the appropriate points in the breath cycle and durations of collection to accumulate the necessary breath sample. Collecting and aggregating smaller sequential breath samples allows the use of compact, energy-efficient diaphragm pumps that are well suited to flow rates needed by this approach. Flow rates requirements using this “sequential sipping approach” (a collection of smaller breath samples) are less than 100 ml/sec for each sip. Sip sequences could be taken over 1-6 ventilator breaths depending on the volume of sample needed for analysis. For example, if only 50 ml were required for analysis, a single 50 ml sip or two 25 ml sips would be adequate to collect the necessary sample. For larger sample requirements, more sips can be accumulated into volumes up to and including 300 ml.

Rapid collection of breath samples during the expiratory phase of ventilation provides isolation of the respiratory tract from incoming dilutive gases that will make samples more difficult to analyze. The expiratory phase of the ventilator cycle can be as short as 1 second. A 0.5 second sample collection duration allows adequate positioning of the collection interval, in time, to collect the breath sample without inspiratory flow overlap at either the beginning or end of the expiratory phase. To collect a sample >50 ml and up to 300 ml over 0.5 seconds requires an approach to rapidly collect this volume. Breath samples can be collected in a rigid vacuum reservoir or plenum containing a flexible breath collection sample bags (i.e., a reservoir) that facilitates rapid sample during the ventilator expiratory cycle. Optionally, a rapidly expandable, vacuum creating reservoir that can be activated to initiate collection of breath samples into a flexible breath collection reservoir connected to a patient's airway.

Breath sample collection can be initiated a number of ways (i) manual initiation of sample collection; (ii) triggered collection of sample using pressure or flow indicative signals from the ventilator circuit or patient airway; (iii) collection triggered by a ventilator circuit or respiratory tract threshold pressure or pressure change measured through: the sampling catheter, endotracheal tube or tracheotomy tube, or pressure or flow sensor in the ventilator circuit; and (iv) automated triggering of the start of breath collection using a trigger from the ventilator circuit that indicates that the ventilator has switched into the expiratory phase of breathing.

Triggering of breath sample collection can be done using other indicators of the inspiration and/or expiration including—pulse oximetry, tri-axial accelerometers, electrocardiograms (ECGs), and two proximal skin electrodes that measure the difference in body surface potential.

Triggered sequencing of breath sample collection:

Sequencing a single breath sample in relation to the ventilator cycle is provided to limit the volume dilution of markers in the breath of patients on ventilation. Such sequencing the collection of breath samples occurs during the expiratory phase of ventilation while airflow into the patient's respiratory anatomy is limited.

Sensing pressure is used to trigger initiation and completion of breath sample collection accumulation over one to many breaths. Pressure can be sensed by a pressure transducer in a breath sampling instrument (described in more detail below) by making continuous measurements of airway pressure and triggering breath sampling based on pressure transitions and or thresholds indicative of the expiratory cycle. Optionally, a silicon strain gauge in or connected to the patient's respiratory tract airway through a catheter lumen can be used to trigger breath sampling. Optionally, measurements of flow in the external ventilator circuit can be used to indicate when the ventilator is in the expiratory part of its ventilation cycle. Optionally, an optical pressure assessment can be used to trigger breath sampling. An optical circuit can be used to detect deformation of an elastic or a movable element in the ventilator airway and react to changes in pressure. Optionally, changes in an ultrasound transmission in a tube or other body exposed to changing pressures can be used to trigger breath sampling.

A signal from the breath sampling device indicating the end of the inspiratory or onset of expiratory phase can be used to trigger breath sampling. Such a signal can be triggered based on: a pulse oximetry signal, motion sensing with tri-axial accelerometer(s), an ECG signal, two proximal electrodes that measure differences in skin surface conduction.

Increasing or controlling the breath sample collection period can also be accomplished by briefly pausing the flow in the ventilator circuit during the expiratory phase to prevent dilution of the sampled breath. Sequencing the collection of breath samples can be performed in relation to the delivery of the drug tracer over a period of time.

As noted, rapid collection of breath samples during the expiratory phase is desired so that there is no, or limited, collection of ambient air during the inspiratory phase of ventilation. During the inspiratory phase of ventilation, the CO₂ levels, both ambient ¹²CO₂ and the metabolized ¹³CO₂ levels are so dilute as to be unmeasurable. The inventors have collected 300 ml breath samples in <0.5 seconds using a vacuum powered system. This timing is much less than the normal expiratory period of 1-3 seconds. Breath sampling has been manually triggered and can be automatically triggered using the different mechanisms described. A ventilator pause can be initiated at the completion of the inspiratory phase as another method of collecting the breath sample without ventilator flow affecting the collected sample CO₂. Using the sequential collection and accumulation of smaller samples in during the baseline and post nebulization collection events, also affords better control of the ventilator circuit pressures and is less likely to cause ventilator circuit pressure drops that might trigger a ventilator leak or PEEP minimum alarm. This has been successfully laboratory tested.

As an alternative breath collection method, longer term scavenging and subsequent release of breath ¹²CO₂ and ¹³CO₂ can be gathered from an expiratory limb of the ventilator circuit. CO₂ gas scavenging can be done using materials like soda-lime (e.g., Sodasorb®). Other materials are designed to scavenge and release compounds like CO₂ using an endothermic reaction to release the scavenged material. This technique could be equally well suited to collection of ¹³CO₂ and ¹²CO₂.

A multi-use breath sample collection chamber can be used to facilitate sequential sampling of breaths for analysis in the detection of respiratory infections and having limited crossover between breath samples. For example, a vacuum reservoir or plenum can be evacuated and pressurized for multiple breath sample collections using a single expandable, elastomeric or laminate breath collection bag for a single patient. A vacuum generated by a pneumatic circuit can be used to evacuate the sample contents of the breath collection bag while the vacuum reservoir is released from vacuum.

Optionally, mechanical compression of a breath sample bag in an instrument plenum can be used to empty the bag of the sample and prepare it for collection of a new sample. Vacuum can be released in a plenum and the plenum then pressurized to push a breath sample out of bag. Optionally, a roller can be used to extrude a breath sample from bag (roller system driven by a mechanism such as linear spring). Optionally, emptying of a breath sample bag can be performed using a manifold to permit wall vacuum to communicate with the internal volume of the breath sample bag between sample collections. Optionally, a vacuum piston can be attached to a plate to squeeze a breath sample out of bag. Optionally, a threaded drive mechanism can be used to close opposing plates on the breath sample bag.

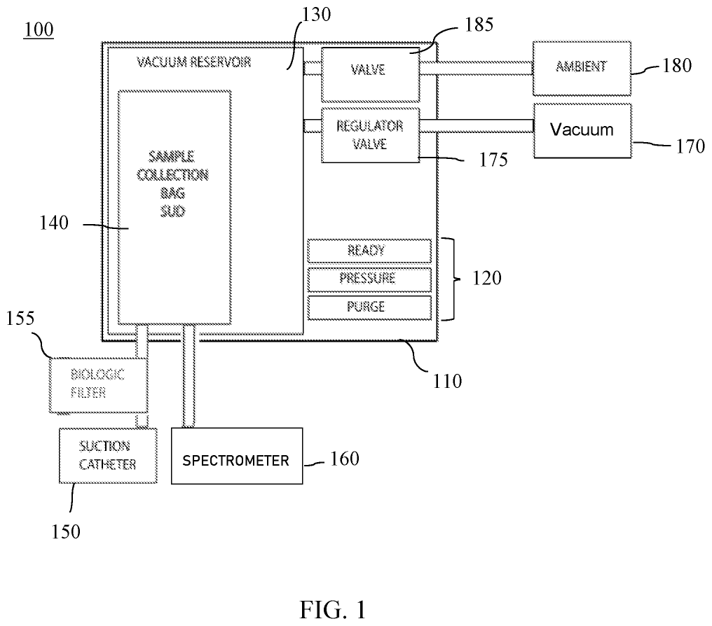

FIG. 1 is a block diagram of a breath sampling device 100 according to an embodiment. As shown in FIG. 1, the breath sample device 100 can include a housing 110, control electronics (not shown), a user interface 120, and a vacuum reservoir 130. The control electronics can include a programmable processor or microcontroller, memory, power supply, sensors, interface connections, and associated wiring. As shown, the user interface 120 can include any configuration of buttons and/or indicators to operate and show the status of the device. For example, the user interface 120 can include buttons and/or indicators for ‘Ready’, ‘Pressure’, and ‘Purge’. Alternatively, the user interface 120 can include a keypad, keyboard, and/or an electronic display (not shown).

The breath sampling device 100 can further include a hard shell, pre-evacuated, vacuum reservoir 130 or plenum for breath sample collection in a breath collection reservoir 140. The breath collection reservoir 140 can be a single use disposable (SUD) bag attached to (i) a suction or dedicated catheter 150 with an in line biological filter 155 and (ii) a spectrometer 160. Collection of a breath sample from a patient is accomplished by opening a normally closed valve/connector that allows the flow of breath into the breath collection reservoir 140 in the evacuated vacuum reservoir 130. The vacuum reservoir 130 can be pre-evacuated during manufacture, or can be actively evacuated by a user during use using a wall vacuum 170 (in a clinical setting) or a stand-alone vacuum pump. Optionally, a spring loaded collection reservoir can be used that, when activated, rapidly expands, drawing a breath sample into its reservoir from the collection catheter 150.

In another embodiment, a collection reservoir can be a stand-alone device in which a breath collection reservoir is placed inside to collect a patient's breath. In this configuration a breath collection reservoir with a separate port to connect to the spectrometer can be used to allow sample transfer to the breath sampling device.

The wall vacuum 170 or a vacuum pump can be used as a pneumatic power source for the rapid collection of breath into an intermediate collection reservoir that the spectrometer can draw the sample from for analysis. The breath collection reservoir 140 can have two valve ports. The valves ports can be on the breath collection reservoir 140 or on a valve manifold that the breath collection reservoir ports connect to. One valve on the breath collection reservoir 140 can be a patient sample collection port—a normally closed port that is attached to the catheter 150 or other mechanism to collect sample breath from the patient's respiratory tract. This valve can be opened when breath collection is initiated, and closed at a prescribed time to adequately fill the breath collection reservoir 140 (>100 ml). The second valve can be a spectrometer collection port—a normally closed port through which the spectrometer 160 retrieves the collected patient breath sample. In situations where the breath sampling device is unable to collect a full patient sample in less than a second, this interim collection and storage apparatus is useful/necessary in ventilator circuits.

Optionally, the breath collection reservoir 140 can have one valve port. In such case, the breath sample and the spectrometer can be accessed via the same port at different times.

The breath collection reservoir 140 or other suitable reservoir can be elastic or easily deformable under the differential forces exerted on it by the vacuum in the vacuum reservoir 130 or compartment, to allow the collection of the patient breath sample which is at positive pressure relative to the vacuum reservoir's vacuum level. A suitable material used to make a SUD bag as the breath collection reservoir 140 is polyvinyl fluoride (e.g., Tedlar®) or another multilayer deformable material that allows the retention of CO₂ gases and bag expansion under the force of an applied vacuum. The SUD bag can be a custom bag, and can be appropriately sized for the application. It has been found that a bag of Tedlar® or other material can hold the breath sample with higher CO₂ content without significant permeation for a maximum period of 10 minutes before analysis of the sample.

Additionally, the SUD bag has a one-way valve to accept a breath sample. This valve allows gas to enter the bag but prevents leakage of the sample prior to the sample being analyzed.

The vacuum reservoir 130 can be a rigid compartment that is accessible, for example via a hinged door, sliding door, etc. to allow placement of the breath collection bags (reservoir) 140 within the vacuum reservoir 130. A single breath collection reservoir 140 can be used for each test or a single breath collection reservoir 140 can be used for each patient to prevent contamination. The rigid vacuum reservoir 130 should be sealed (i.e., using a gasket, 0-ring, or another suitable mechanism) to allow the opening and closing of the vacuum reservoir 130 around the breath collection reservoir 140 and its ports so that adequate vacuum can be achieved in the vacuum reservoir 130 to facilitate filing the breath collection reservoir 140. A hard vacuum and perfect sealing is not necessary for the operation of this system.

Such a rigid vacuum reservoir 130 or compartment can be a single use disposable device or a portion of an integrated instrument. The vacuum reservoir 130 can be attached through a pressure regulator valve 180 or a pulsing on/off valve (electronically or mechanically actuated to prevent filling of the breath sample breath collection reservoir 140 before being ready) to the vacuum 170. The vacuum 170 can be from a wall vacuum receptacle located in a patient's room or other vacuum source capable of providing continuous vacuum establishing an adequate plenum vacuum level to support rapid sample collection within the expiratory period of ventilation, such as a vacuum pump. If the vacuum source or pressure is insufficient for a proper, regulated collection of breath samples, the control electronics in the breath sample collection device 100 can sense this pressure condition (measuring the pressure in relation to a programmed threshold) and can prevent sample collection, and can alert the clinician user.

The rate of filling the breath collection reservoir 140 is related to the vacuum pressure in the vacuum reservoir 130. The breath collection reservoir 140 filling time is inversely related to the vacuum pressure in the vacuum reservoir 130. Depressurizing the vacuum reservoir 130 will cause a breath sample to be rapidly collected in the breath collection reservoir 140. The breath sample is brought to the breath collection reservoir 140 through an indwelling catheter or other connection to the patient's respiratory tract such as an endotracheal tube with sample collection ports.

Depressurizing the vacuum reservoir 130 can be initiated in a number of ways, for example, (i) via a manual or electronically mediated valve opening by a clinician observing the patient's inspiration and expirations (breathing) and timed so that the breath sample is collected when the patient is exhaling; (ii) pressure triggered evacuation of the vacuum reservoir 130 while monitoring the airway pressure to trigger evacuation of the vacuum reservoir 130 and opening of the breath collection reservoir causing filling when exhalation has begun; (iii) a ventilator signal triggering evacuation of the vacuum reservoir 130; (iv) triggering by pulse oximetry, tri-axial accelerometers, electrocardiograms (ECGs), and/or two proximal skin electrodes that measure the difference in body surface potential caused by respiration. Generally, the vacuum reservoir 130 is opened to the ambient atmospheric pressure 180 via a value 185 to relieve the vacuum so that the vacuum reservoir 130 can be accessed to retrieve/replace the breath collection reservoir 140.

The breath collection reservoir 140 can have a second port through which the breath sample is retrieved by the spectrometer sample collection port. Alternatively, breath samples can be collected from the breath collection reservoir 140 or reservoir using the same inlet port and an electronic valve system that directs the flow to and from the breath collection reservoir 140. The breath collection reservoir 140 can be a one time or multiple use reservoir for collection of the breath samples. The port to which the catheter 150 is connected can also serve as a port for the transfer of the collected sample to the spectrometer 160 if stasis and flow redirection valves are located between the sample collection container and the catheter port. This port can also be used as a link to vacuum clearance of the sample collection chamber(s).

For example, in an embodiment, a pneumatic manifold 210 including a series of valves (shown within the dash lined boxes) can be used to facilitate gas flow as shown in FIG. 2. FIG. 2 shows a 0.2-micron biologic filter 255 filtering breathing from a suction/sampling catheter 257 attached to patient P into the pneumatic manifold 210. The pneumatic manifold 210 includes multiple valves and routing including a vacuum valve 215, a vacuum plenum ventilation release valve 220 to release vacuum from the vacuum plenum 230, sample collection valves 222 and a spectrometer valve 224. The spectrometer valve 224 can be connected to a port for connection to a spectrometer to a spectrometer 290 integrated into the breath collection device. In conjunction with the sample collection valves 222, the spectrometer valve 224 is configured to connect the baseline breath collection reservoir 240 and the sample breath collection reservoir 245 to the spectrometer 290.

The pneumatic manifold 210 can include 1 to n number of sample collection valves ports 222 that can be connected to breath collection reservoirs 240 and 245. Sample container connection detectors 250 are connected to the control electronics and provide a control signal related to whether a breath collection reservoir 240, 245 is connected or not to a sample collection valve port 222. The breath collection reservoir (Bₙ) 240 is for a baseline breath and the breath collection reservoir (Sᵣ) 245 is for a sample breath. A vacuum reservoir pressure (VRP) sensor 262 provides signals to the control electronics related to the pressure in the vacuum reservoir 230 to assist in operating the valves assuring adequate vacuum in the reservoir, >120 cm H₂O vacuum. A catheter pressure sensor 264 can be located in the patient's respiratory airway and connected to the control electronics to sense the patient's breathing or ventilator pattern. This allows for accuracy of breath collection timing.

Method of breath collection:

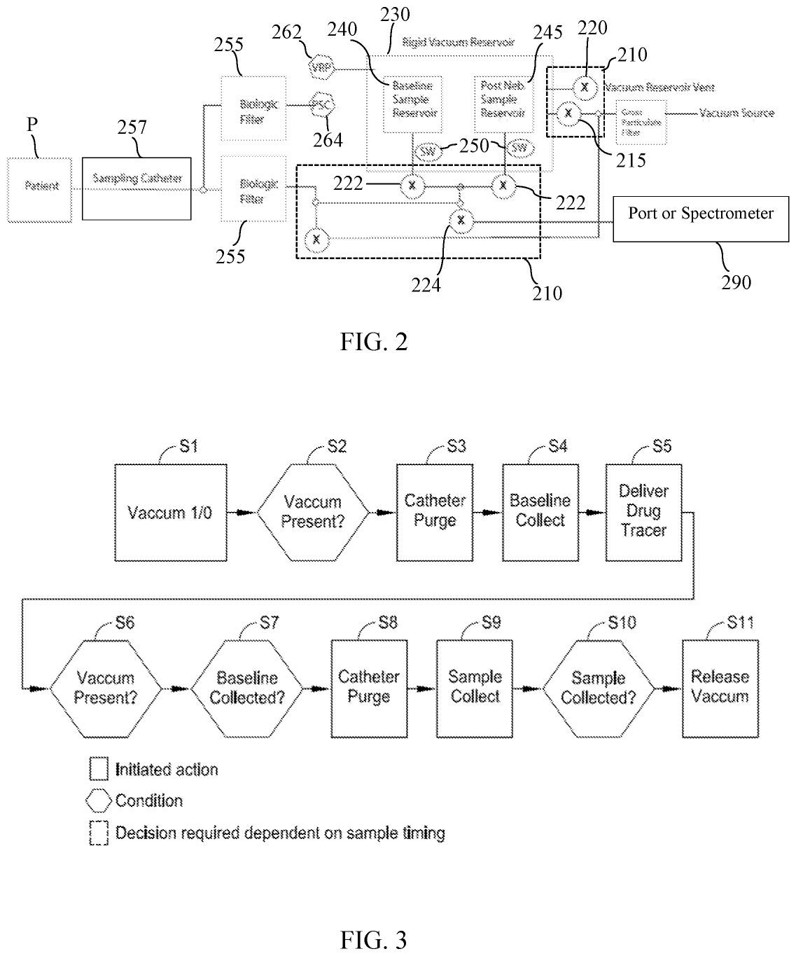

FIG. 3 is a flowchart showing exemplary steps in a breath sample collection operation of a breath sampling system according to an embodiment. Button initiated actions can be performed manually by a user or automatically by the control electronics. Prior to operation, if in use for the patient, wall suction or another vacuum source is detached from a suction catheter, the breath sampling device is attached to the wall suction or vacuum pump and the patient's catheter or nebulizer if the patient is ambulatory. The wall vacuum should be set to 150 mm Hg for a breath collection bag fill time of approximately 1 second.

At step S1, a vacuum interface is initiated, and the control electronics determines if a vacuum is present in step S2. If a vacuum is not present, the process does not proceed. Once a vacuum is detected, the patient's catheter is purged in step S3 in preparation for collection of a baseline breath sample. In step S4, a collection of a baseline breath sample is initiated. In step S5, a drug tracer is delivered to the patient. In step S6, the control electronics again determines if a vacuum is present and, in step S7, the control electronics determines if a baseline breath sample has been collected and the drug has been delivered. If a vacuum is not present in S6 or a baseline breath sample has not been collected, the process does not proceed. Once a vacuum and baseline breath sample have been verified, the catheter is purged in step S8 in preparation for collection of a breath sample to be compared to the baseline breath sample. In step S9, a collection of a breath sample to initiated and collection of the breath sample is verified in step S10. In step S11, the vacuum in the vacuum reservoir is released so the breath collection reservoirs 240, 245 can be removed and transferred to the spectrometer for analysis or the breath collection reservoirs replaced anew.

It should be understood that the foregoing description is only illustrative of the present invention. Various alternatives and modifications can be devised by those skilled in the art without departing from the present invention. Accordingly, the present invention is intended to embrace all such alternatives, modifications, and variances that fall within the scope of the appended claims.

What is claimed is:

1. A breath collection system, comprising:
a vacuum reservoir;
a vacuum connected to the vacuum reservoir;
a breath collection reservoir configured to be located inside the vacuum reservoir during a breath collection and removed after a breath is collected from a patient; and
a patient interface device connected to the breath collection reservoir to collect the breath from the patient.

2. The breath collection system of claim 1, further comprising a housing that houses the vacuum reservoir and the breath collection reservoir.

3. The breath collection system of claim 2, wherein the vacuum is a vacuum pump in the housing.

4. The breath collection system of claim 1, further comprising a user interface.

5. The breath collection system of claim 1, further comprising a biologic filter between the patient interface and the breath collection reservoir.

6. The breath collection system of claim 1, wherein the patient interface includes a nebulizer.

7. The breath collection system of claim 1, wherein the patient interface includes a catheter.

8. The breath collection system of claim 1, further comprising a regulator valve between the vacuum and the vacuum reservoir to regulate pressure in the vacuum reservoir.

9. The breath collection system of claim 1, wherein the breath collection reservoir is a flexible bag.

10. The breath collection system of claim 1, wherein the breath collection reservoir is a plurality of breath collection reservoirs.

11. The breath collection system of claim 10, wherein the plurality of breath collection reservoirs includes a baseline breath collection reservoir and a sample breath collection reservoir.

12. The breath collection system of claim 1, further comprising a pneumatic manifold between the vacuum and the vacuum reservoir and between the patient interface device and the breath collection reservoir.

13. The breath collection system of claim 12, further comprising a pressure sensor in the vacuum reservoir.

14. A breath collection device, comprising:
a vacuum reservoir;
a baseline breath collection reservoir and a sample breath collection reservoir both configured to be located inside the vacuum reservoir and removed after breath samples are collected;
a pneumatic manifold including a plurality of valves;
a patient interface device to collect the breath from a patient connected to the baseline breath collection reservoir and the sample breath collection reservoir via a corresponding sample collection valve in the pneumatic manifold; and
a vacuum connected to the vacuum reservoir via a vacuum valve in the pneumatic manifold.

15. The breath collection device of claim 14, further comprising a pressure sensor in the vacuum reservoir.

16. The breath collection device of claim 14, wherein the plurality of valves includes 1 to n number of sample collection valves, and the 1 to n sample collection valves are connected to a corresponding baseline breath collection reservoir or a sample breath collection reservoir.

17. The breath collection device of claim 14, further comprising a spectrometer valve in the pneumatic manifold and configured to direct the collected breath to a spectrometer.

18. The breath collection device of claim 17, wherein the spectrometer valve is configured to connect the baseline breath collection reservoir and the sample breath collection reservoir to the spectrometer.
